(19) United States
(12) Patent Application Publication
(10) Pub. No.: US 2005/0273871 A1
(43) Pub. Date: Dec. 8, 2005

(54) Method for diagnosing arrhythmogenic right ventricular dysplasia

(76) Inventors: Yoshihiro Asano, Osaka (JP); Seiji Takashima, Osaka (JP); Masafumi Kitakaze, Osaka (JP)

Correspondence Address: STERNE, KESSLER, GOLDSTEIN & FOX PLLC, 1100 NEW YORK AVENUE, N.W., WASHINGTON, DC, 20005, US

(21) Appl. No.: 10/860,601

(22) Filed: Jun. 4, 2004

(51) Int. Cl. C12Q 1/68; A01K 67/027

(52) U.S. Cl. 800/18; 435/6

(57) ABSTRACT

A specific strain of KK obese mice was found to show a phenotype peculiar to human arrythomogenic right ventricular dysplasia (ARVD), and Lamrl-functional transposon 1 (Lamr1-tp1) was determined to be responsible for this phenotype. Furthermore, the translation product of Lamr1-tp1 was shown to interact with HP1-alpha. Together with the knowledge that human ARVD loci are reported to exist close to the retroposons of Lamr1 or histone-modulating protein genes, aberrant interaction of mutant-LAMR1 and HP1-alpha seems to be a cause of ARVD. Thus, the present invention relates to methods for diagnosing ARVD. The present invention further relates to an animal model of ARVD, and cells transfected with mutant Lamr1 gene demonstrated to cause ARVD in the animal model. Moreover, the present invention relates to methods of screening for compounds suppressing ARVD using the animal model or the transfected cells.

### FIELD OF THE INVENTION

The present invention relates to methods for diagnosing arrhythmogenic right ventricular dysplasia (ARVD). Furthermore, the present invention relates to an animal model of ARVD, and cells transfected with a mutant Lamr1 gene that causes ARVD in the animal model. Moreover, the present invention relates to methods of screening for compounds suppressing ARVD using the animal model or the transfected cells.

### BACKGROUND OF THE INVENTION

Arrhythmogenic right ventricular dysplasia (ARVD) is a hereditary cardiomyopathy that often causes sudden death in the young. ARVD is characterized by the gradual loss of cardiomyocytes and compensatory replacement with either adipose or fibrous tissue. ARVD is now established as a major cause of sudden cardiac death among the juvenile population and athletes, but the difficulty of making a diagnosis before the onset of cardiac events still remains to be overcome. In Italy, ARVD accounts for 20% of all sudden deaths in individuals under 35 years old and 22% of sudden deaths in athletes (Thiene, G. et al., Trends Cardiovasc. Med. 7: 84-90 (1997)). However, the etiology of this disease is still unknown. In about 30% of ARVD patients, familial occurrence is reported. Six loci have already been mapped (ARVD, 14q23 (Thiene, G. et al., Trends Cardiovasc. Med. 7: 84-90 (1997)); ARVD2, lq42 (Thiene, G. et al., Trends Cardiovasc. Med. 7: 84-90 (1997)); ARVD3, l4ql2 (Severini, G. M. et al., Genomics 31: 193-200 (1996)); ARVD4, 2q32 (Rampazzo, A. et al., Genomics 45: 259-263 (1997)); ARVD5, 3q23 (Ahmad, F. et al., Circulation 98: 2791-2795 (1998)); and ARVD6, 10q12 (Li, D. et al., Am. J. Hum. Genet. 66: 148-156 (2000)), but the only gene identified so far is ARVD2, which corresponds to the cardiac ryanodine receptor gene and causes a condition with different features from the other forms of ARVD (Tiso, N. et al., Hum. Mol. Genet. 10: 189-194 (2001)). As in humans, a naturally occurring phenotype of right ventricular dysplasia (RVD) has been reported in dogs, cats, and minks (Bright, J. M. and McEntee, M., J. Am. Vet. Med. Assoc. 207: 64-66 (1995); Simpson, K. W. et al., J. Vet. Intern. Med. 8: 306-309 (1994); Fox, P. R. et al., Circulation 102: 1863-1870 (2000); Ishikawa, S. et al., Arch. Pathol. Lab. Med. 101: 388-390 (1977)).

Laminin receptor 1 (LAMR1) is a ribosomal protein localized in the nucleus and is known to be involved in apoptosis (Sato, M. et al., Biochem. Biophys. Res. Commun. 229: 896-901 (1996); Kaneda, Y. et al., Cell Death Differ, 5: 20-28 (1998)). Genomic databases indicate the existence of up to 40 and 32 retroposons of Lamr1 in humans and mice, respectively. Lamr1 gene family in mammals, except the functional locus, is suggested to be entirely composed of retrotransposons and processed pseudogenes. Most of the processed retroposons are not expressed and lack functional activity. However, several active retrotransposons and pseudogenes have been identified (McCarrey, J. R. and Thomas, K., Nature 326: 501-505 (1987); Gebara, M. M. and McCarrey, J. R., Mol. Cell. Biol 12: 1422-1431 (1992); Dahl, H. H. et al., Genomics 8: 225-232 (1990); Hirotsune, S. et al., Nature 423: 91-96 (2003)). In human, a highly conserved mutant-Lamr1 has been isolated from a fetal brain cDNA library (Richardson, M. P. et al., Gene 206: 145-150 (1998)).

### SUMMARY OF THE INVENTION

The present inventors found mice that inherit right ventricular dysplasia (RVD), and identified a mutation in the laminin receptor 1 (Lamr1) gene responsible for the occurrence of RVD in these mice. When the mutant Lamr1 gene was introduced into normal mice by breeding or direct injection, RVD similar to that in the RVD mice was caused. Furthermore, according to an in vitro study of cardiomyocytes expressing the gene, early cell death accompanied by alteration in the chromatin architecture was observed. Moreover, heterochromatin protein 1 (HP1) was determined to bind to the protein encoded by the mutant gene. HP1 is known as a dynamic regulator of heterochromatin sites. Thus, the binding of mutant-LAMR1 suggests that it impairs a critical process in transcriptional regulation. Indeed, via gene chip analysis, mutant-LAMR1 was discovered to cause specific changes in gene expression in cardiomyocytes. Therefore, the present inventors concluded that the product of the Lamr1 retroposon interacts with HP1 to cause degeneration of cardiomyocytes, which mechanism may also contribute to the etiology of human ARVD.

Thus, the present invention provides;

[1] A method for diagnosing arrhythmogenic right ventricular dysplasia (ARVD), which comprises the steps of:
(1) detecting mutation in the amino acid sequence of a protein encoded by laminin receptor 1 (Lamr1) gene or retroposon thereof in a subject; and
(2) when a mutation is found in step (1), determining the subject to be susceptible to ARVD.

[2] The method of [1], wherein the mutation in the amino acid sequence of the protein encoded by Lamr1 gene is determined based on the nucleotide sequence of the genomic Lamr1 gene of the subject.

[3] The method of [1], wherein the mutation in the amino acid sequence of the protein encoded by Lamr1 gene or retroposon thereof is determined based on the nucleotide sequence of the expressed mRNA of the subject.

[4] The method of [1], wherein the mutation in the amino acid sequence of the protein encoded by Lamr1 gene or retroposon thereof is determined based on the amino acid sequence of the expressed Lamr1 protein (LAMR1) of the subject.

[5] The method of [1], wherein the mutation in the amino acid sequence of the protein encoded by Lamr1 gene or retroposon thereof is determined by detecting the binding of the expressed LAMR1 of the subject to heterochromatin protein 1 alpha (HP1-alpha), and when binding of LAMR1 to HP1-alpha is detected, the amino acid sequence of protein encoded by the Lamr1 gene is determined to contain mutation.

[6] A method for diagnosing ARVD, which comprises the steps of:
(1) detecting a mutation in the amino acid sequence of protein encoded by Hp1-alpha gene of a subject; and
(2) when a mutation is found in step (1), determining the subject to be susceptible to ARVD.

[7] The method of [6], wherein the mutation in the amino acid sequence of the protein encoded by Hp1-alpha gene is determined based on the nucleotide sequence of the genomic Hp1-alpha gene of the subject.

[8] The method of [6], wherein the mutation in the amino acid sequence of the protein encoded by Hp1-alpha gene is determined based on the nucleotide sequence of the expressed mRNA of the Hp1-alpha gene of the subject.

[9] The method of [6], wherein the mutation in the amino acid sequence of the protein encoded by Hp1-alpha gene is determined based on the amino acid sequence of the expressed HP1-alpha of the subject.

[10] The method of [6], wherein the mutation in the amino acid sequence of the protein encoded by Hp1-alpha gene is determined by detecting the binding of the expressed HP1-alpha of the subject to LAMR1, and when binding of the Hp1 protein to LAMR1 is detected, the amino acid sequence of the protein encoded by Hp1-alpha gene is determined to contain mutation.

[11] A diagnostic agent for ARVD, which comprises at least a substance that enables the detection of a mutation in the amino acid sequence of the protein encoded by Lamr1 gene of a subject compared to a naturally occurring sequence.

[12] The diagnostic agent of [11], which comprises an antibody against a mutant LAMR1 as the substance.

[13] The diagnostic agent of [11], which comprises HP1-alpha as the substance.

[14] The diagnostic agent of [11], which comprises a probe against Lamr1 gene as the substance.

[15] The diagnostic agent of [11], which comprises primers that can be used for specifically amplifying Lamr1 gene as the substance.

[16] A diagnostic agent for ARVD, which comprises at least a substance that enables the detection of a mutation in the amino acid sequence of the protein encoded by Hp1-alpha gene of a subject compared to a naturally occurring sequence.

[17] The diagnostic agent of [16], which comprises an antibody against a mutant HP1-alpha as the substance.

[18] The diagnostic agent of [16], which comprises LAMR1 as the substance.

[19] The diagnostic agent of [16], which comprises a probe against Hp1-alpha gene as the substance.

[20] The diagnostic agent of [16], which comprises primers that can be used for specifically amplifying Hp1-alpha gene as the substance.

[21] An animal model of ARVD, expressing a mutant Lamr1 gene.

[22] The animal model of [21], wherein ARVD is caused by the injection of a mutant Lamr1 gene into a ventricle.

[23] The animal model of [22], wherein the gene is injected into the right ventricle.

[24] The animal model of [21], wherein the animal is a transgenic animal expressing a mutant Lamr1 gene in a ventricle.

[25] The animal model of [24], wherein the mutant Lamr1 gene is expressed in the right ventricle.

[26] The animal model of [21], wherein the animal is a mouse.

[27] A transgenic non-human animal transduced with a polynucleotide that expresses a mutant Lamr1 gene.

[28] The transgenic non-human animal of [27], wherein the animal is a rodent.

[29] The transgenic non-human animal of [28], wherein the animal is a mouse.

[30] The transgenic non-human animal of [27], wherein the animal shows symptoms of ARVD.

[31] The transgenic non-human animal of [27], wherein the mutant Lamr1 gene is Lamr1-tp1.

[32] The transgenic non-human animal of [27], wherein the animal expresses the Lamr1 gene in a ventricle.

[33] The transgenic non-human animal of [27], wherein the animal expresses the Lamr1 gene in the right ventricle.

[34] A cell expressing a mutant Lamr1 gene.

[35] The cell of [34], wherein the cell is a cardiomyocyte.

[36] A method of screening for a compound using the animal of [21] or [27], which comprises the steps of:
(1) administering a test compound to the animal of [21] or [27];
(2) detecting symptoms of right ventricular dysplasia (RVD) in the animal;
(3) selecting the compound detected as alleviating or suppressing a symptom of RVD.

[37] A method of screening for a compound using the cell of [34], which comprises the steps of:
(1) culturing the cell of [34] in the existence of a test compound; and
(2) selecting the compound that prolongs the life of the cell or impedes changes in the chromatin architecture compared to a cell cultured in the absence the test compound.

[38] A method of screening for a compound using the cell of [34], which comprises the steps of:
(1) culturing the cell of [34] in the existence of a test compound; and
(2) selecting the compound that suppresses changes in gene expression due to the introduction of Lamr1-tp1.

[39] A method of screening for a compound using mutant-LAMR1 that binds to HP1-alpha and HP1-alpha, which comprises the steps of:
(1) contacting the mutant-LAMR1 and HP1-alpha in the presence of a test compound; and
(2) selecting the compound that inhibits binding between the mutant-LAMR1 and HP1-alpha.

### BRIEF DESCRIPTION OF THE DRAWINGS

FIG. 1 shows photographs and electrocardiography showing the heart of an 8-week-old KK/Rvd mouse wherein the outer side of right ventricle wall is covered by massive fibrosis that never extends to the left ventricle like human ARVD. (a) to (d) show macroscopic view of the heart of the 8-week-old KK/Rvd mouse: (a) front view; (b) back view; and (c and d) sagittal section of the heart, wherein the arrow heads indicate the degenerated area of the right ventricle. (e and f) show high magnification of stained degenerated area boxed in (d): (e) result of HE staining; and (f) result of Masson-trichrome staining. The arrows in (e) indicate the cardiomyocytes dense stained by eosin; and arrows in (f) indicate the degraded cardiomyocytes surrounded by fibrous tissue. (g) is a photograph depicting the result of anti-MCP-1 antibody staining, showing infiltration of macrophages into degraded area. (h) is the electrocardiography of the 8-week-old KK/Rvd mouse showing prolonged QRS duration. Symbols in the figure indicate: RV, right ventricle; LV, left ventricle; RA, right atrium; LA, left atrium; and *, indicates calcification. Scale bar: (a to c), 3 mm; (e and f), 50 μm; and (g), 10 μm.

FIG. 2 shows schematic illustrations depicting the location, alignment and expression of Lamr1-tp1. (a) is a schematic illustration of chromosome 7. The rvd locus was mapped on mouse chromosome 7 adjacent to D7Mit270. (b) is a schematic illustration of Lamr1-tp1, A 1031 bp-long sequence was inserted in the genome of KK/Rvd mouse but not in PWK mouse. (c) is a schematic illustration of the alignment of Lamr1 retroposons in the mouse genome database. The amino acid sequences encoded by the Lamr1 retroposons are compared to that of LAMR1 (original Lamr1 gene located on chromosome 9). Arrows indicate positions of mutated amino acids.

FIG. 3 shows the result of experiment to specify the tissue expressing Lamr1-tp1 in KK/Rvd mouse. The transcription of Lamr1-tp1 was confirmed only in KK/Rvd heart, liver and skeletal muscle. (a) RT(−) indicates the result with RNA samples from KK/Rvd tissues before reverse transcription polymerase chain reaction (RT-PCR) as the PCR template. Primers designed to amplify either Lamr1 or Lamr1-tp1 specific mutated sequence were used for the PCR. (b) RT(+) indicates the result of PCR with cDNA samples after RT-PCR as the PCR template and the same primers as in (a). (c) The same template as in (b) was used with reaction primers designed in the intron region of Lamr1 or up-or-downstream of Lamr1-tp1 gene for PCR. (d) PCR products of (b) were digested with restriction enzyme NheI that specifically recognizes a site only in Lamr1-tp1.

FIG. 4 shows photographs depicting the result of direct Lamr1-tp1 gene injections into heart of C57BL/6 mice. Lamr1-tp1 specific degradation of the myocardium was caused by the injection. Following GFP co-expression plasmids were injected: (a) to (c), pIRES2EGFP-Lamr1-tp1; and (d) to (f), pIRES2EGFP-Lamr1. In (a) and (d), the expression of GFP was detected at the gene injected area. (b) and (e) show macroscopic view, and (c) and (f) magnified view of the transfected sites.

FIG. 5 shows the result of transformation of Lamr1-tp1 or Lamr1 gene into mice. (a) and (b) are photographs showing the result of expression analysis (Northern blot) in transgenic mouse models. Both transgenes, Lamr1-tp1 and Lamr1, were equally detected by this analysis. (a) depicts a photograph showing the expression of transgenes in the heart among transgenic mouse models; Lamr1-tp1 Lines 1 and 2, transfected with PBS-alphaMHC-Lamr1-tp1; Lamr1 Lines 1 and 2, with PBS-alphaMHC-Lamr1; non-TG, non-transgenic strain; and WT, wild-type strain. (b) depicts photographs showing the systemic expression of Lamr1-tp1 in transgenic mouse models transfected with PKSCX-Lamr1-tp1. (c) depicts a macroscopic view of PBS-alphaMHC-Lamr1-tp1 transfected 10-week-old mouse heart. A global change in the right ventricle was detected.

FIG. 6 shows the result of in vitro expression of Lamr1-tp1 and Lamr1 genes in cardiomyocytes. Lamr1-tp1 caused cell death of the cardiomyocytes. (a) is a graph showing viability of the cardiomyocytes infected with recombinant adenovituses expressing Lamr1-tp1 or Lamr1 at the indicated MOI. 48 hr after adenovirus infection, cells expressing Lamr1-tp1 had lower MTS activity. *p<0.05 vs. Lamr1, **p<0.005 vs. Lamr1. (b) depicts photographs depicting GFP and actin expression in the cardiomyocytes assayed 8 hr after adenovirus infection. Cell numbers of cardiomyocytes transfected with Lamr1-tp1 and Lamr1 were equivalent at this time point. The ratio of densitonetric measurement of GFP/actin is indicated below. (c) depicts photographs showing staining of the nuclei of rat cardiomyocytes expressing Lamr1-ires-GFP or Lamr1-tp1-ires-GFP with anti LAMR1 antibody FD4818 (middle) or DAPI (left). LAMR1 staining showed perfect mirror image against DAPI positive area. LAMR1-TP1 was translocated and partially overlapped with DAPI positive area. Scale bar, 20 μm.

FIG. 7 shows the interaction of LAMR1-TP1 with HP1-alpha. (a) is a photograph showing co-immunoprecipitation of RVAP27 with LAMR1-TP1 but not with LAMR1 in COS7 cells labeled by ³⁵S-cystein and methionine. (b) depicts photographs showing the result of separation of the complex of radiolabeled LAMR1-TP1 and RVAP27 on a phenyl reverse phase column. (c) is a photograph showing the silver-stained final purification product of RVAP27. (d) shows the amino acid sequence of RVAP27. In the amino acid sequence, “WKDTDEADLVLA” and “CPQIVIAFYEER” were confirmed to match with those of human HP1-alpha by direct N-terminal sequencing, and “SNFSNSADDIK” was confirmed to match with those of human HP1-alpha by liquid chromatography-mass spectrometry/mass spectrometry. Cystein was detected as Cys-S-propionamide. (e) depicts photographs showing the co-immunoprecipitation of V5-tagged LAMR1-TP1 expressed in COS7 cells with myc-tagged HP1-alpha by anti-myc antibody, V5-tagged LAMR1 was not co-immunoprecipitated with myc-tagged HP1-alpha by anti-myc antibody. V5-tagged LAMR1-TP1 nor LAMR1 did co-immunoprecipitate with IgG derived from non-immunized serum. (f) depicts photographs showing the staining of rat cardiomyocyte with anti-HP1-alpha antibody. HP1-alpha co-localizated to the DAPI positive heterochromatin area.

FIG. 8 shows the changes of gene expression induced by LAMR1-TP1. Expressional changes among control (non-transfected), Lamr1-tp1, and Lamr1 transfected neonatal mouse cardiomyocytes were detected in duplicate using Affymetrix arrays. Fold-changes reflect those listed in the original data files. Genes showing big difference in LBP/p40rvd expression and LBP/p40t expression are listed in (b). (a) is a graph showing the normalized intensity of fold changes 12 hr after the transfection. (b) Fold-changes of genes at 6, 12, and 24 hr after the transfection are listed: upper column, genes increased in Lamr1-tp1 transfected cells; and lower column, genes decreased in Lamr1-tp1 transfected cells. (c and d) is a graph showing the fold-changes in a subset of these genes 12 hr after the transfection validated by quantitative Real-Time PCR: (c), LRG-21; and (d), TDAG51. LRG-21, activating transcription factor 3; TDAG51, pleckstrin homology-like domain, family A, member 1; MIC-l, growth differentiation factor 15; RNP, ribonucleic acid binding protein S1; BNP, brain natriuretic peptide; GGPP, geranylgeranyl diphosphate synthase 1; and TDD5, N-myc downstream regulated-like.

### DETAILED DESCRIPTION OF THE INVENTION

The words “a”, “an” and “the” as used herein mean “at least one” unless otherwise specifically indicated.

Arrhythmogenic Right Ventricular Dysplasia Diagnosis

According to the present invention, mutation in the laminin receptor 1 (Lamr1) gene was found to be responsible for the occurrence of RVD in mice. Furthermore, heterochromatin protein 1 alpha (HP1-alpha) was determined to bind to mutant-Lamr1 protein (mutant-LAMR1) encoded by the gene. HP1 is known as a dynamic regulator of heterochromatin sites. Thus, the binding of mutant-LAMR1 suggests that it impairs a critical process in transcriptional regulation. Indeed, via gene chip analysis, mutant-LAMR1 was discovered to cause specific changes in gene expression in cardiomyocytes. Therefore, the present inventors concluded that the product of the mutant-LAMR1 interacts with HP1-alpha to cause degeneration of cardiomyocytes. Together with the knowledge that human ARVD loci are reported to exist close to the retroposons of Lamr1 or histone-modulating protein genes (Table 1), the present inventors determined that arrhythmogenic right ventricular dysplasia (ARVD) patients can be diagnosed by detecting mutant LAMR1 expression or HP1-alpha mutation.

Thus, the present invention provides methods for diagnosing ARVD. Specifically, according to an aspect of the present method, ARVD is diagnosed in a patient by detecting mutation in the amino acid sequence of the expressed protein encoded by a laminin receptor 1 (Lamr1) gene or a retroposon thereof in a subject. According to the method, a subject detected to express a mutant Lamr1 protein (mutant-LAMR1) encoded by a mutant Lamr1 gene or a retroposon thereof in a ventricular (particularly right ventricular) is diagnosed to be susceptible to ARVD.

The original Lamr1 gene encodes a ribosomal protein localized in the nucleus, and consists of 7 exons and 6 introns located on chromosome 9. A retroposon of 1031 bp mutant-Lamr1 gene was found as the cause of ARVD in mice. This retroposon was dubbed “laminin receptor 1, transposed paralog 1 (Lamr1-tp1)”. The protein encoded by this Lamr1-tp1 gene is a preferred example of the mutant-LAMR1 to be detected in the diagnosis of the present invention. However, the mutant-LAMR1 is not restricted to those encoded by Lamr1-tp1 gene. According to the present invention, HP1-alpha was shown to bind with Lamr1-tp1 protein (LAMR1-TP1) but not with LAMR1. This binding of LAMR1-TP1 to HP1-alpha was demonstrated to influence transcriptional regulation of genes that are essential for the survival of cardiomyocytes, finally leading to lethal cell dysfunction. Thus, any mutations in a gene that leads to the expression of a mutant-LAMR1 that binds to HP1-alpha and influences the transcriptional regulation of genes that are essential for the survival of cardiomyocytes can be detected in the present diagnosis method.

The expression of mutant-LAMR1 in a subject can be detected, for example, by detecting mutation in the amino acid sequence of protein encoded by Lamrl gene or retroposon thereof in the subject compared to a naturally occurring sequence. Specifically, the mutation in the amino acid sequence of protein encoded by Lamrl gene can be determined based on the nucleotide sequence of the genomic Lamr1 gene or expressed mRNA, or the amino acid sequence of expressed LAMR1 protein in the subject.

According to another aspect of the present invention, ARVD is diagnosed in a patient by detecting mutation in the amino acid sequence of expressed protein encoded by Hp1-alpha gene of a subject compared to a naturally occurring sequence. Through the method, a subject detected to express a mutant HP1-alpha encoded by an altered Hp1-alpha gene that binds to LAMR1 in the ventricular is diagnosed to be susceptible to ARVD.

The expression of a mutant HP1-alpha can be detected, for example, by detecting mutation in the amino acid sequence of protein encoded by Hp1-alpha gene based on the nucleotide sequence of the genomic or expressed mRNA of the Hp1-alpha gene, or the amino acid sequence of expressed HP1-alpha in the subject.

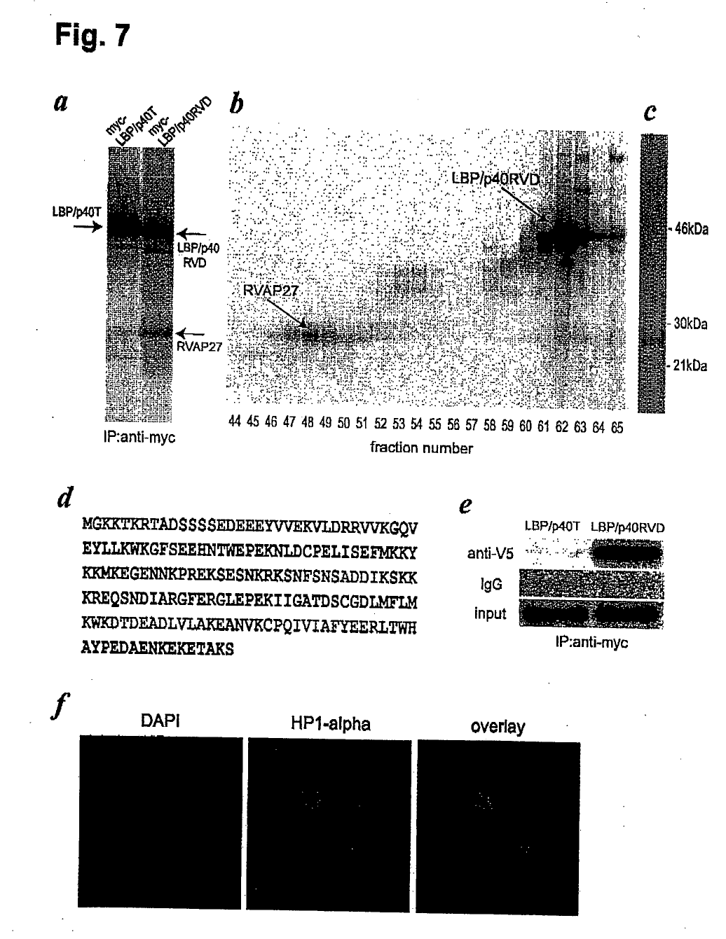

The detection of sequence mutation is known in the art and any conventional methods may be used for the diagnosis of the present invention. Specifically, a mutation can be confirmed by comparing the sequence of the expressed Lamr1 or Hp1 gene in a subject to that of a corresponding naturally occurring sequence. Herein, the phrase “naturally occurring sequence” refers to a sequence of Lamr1 or Hp1-alpha gene found in normal healthy subjects, and is also referred to as the native Lamr1 or Hp1 gene or wild-type Lamr1 or Hp1 gene. The nucleotide sequences of human and mouse Lamr1 and Hp1 genes have been reported together with their coding amino acid sequences (Proc. Natl. Acad. Sci. USA 99: 16899-16903 (2002)). Further, the sequences are also submitted to GenBank (e.g., human Lamrl gene: GenBank Accession No. BC018867: mouse Lamr1 gene: GenBank Accession No. AF140348; human Hp1 gene: GenBank Accession No. BC006821; mouse Hpl gene: GenBank Accession No. AF216290). The amino acid sequence of native HP1-alpha is shown in FIG. 7d.

For example, based on the known nucleotide sequences of Lamr1 and Hp1-alpha genes probes or primers can be constructed for detecting their genomic sequence or mRNA. For example, using a probe against the mutant genes (mutant Lamr1 or mutant Hp1-alpha genes), Northern blot hybridization may be conducted to detect their mRNAs. Such hybridization of the probe to a transcript of the genes may be also performed on a DNA array to readily detect the expression of plurality of genes (e.g., mutant and/or naturally occurring Lamr1 and Hp1-alpha genes, and, if needed, other genes as control). The detection may be also carried out be amplifying the mutant genes using primers to the mutant gene via RT-PCR, etc. The detection may also be carried out using a probe that hybridizes to both the mutant gene and the naturally occurring gene; or primers that amplify both the mutant gene and the naturally occurring gene. When such probes or primers that are not specific to the mutant genes are used, the sequence of hybridized or amplified polynucleotides may be determined via conventional sequencing methods for detecting the mutation in the amino acid sequence of the LAMR1 or HP1-alpha expressed in a subject.

Furthermore, the expressed proteins (LAMR1 or HP1-alpha) may be detected for the present diagnosis through immunoassay methods. Specifically, antibodies specifically recognizing mutant proteins may be used to detect the expression of the mutant proteins in a ventricle of a subject. It is important to use an antibody that distinguishes between the naturally occurring protein and the mutant protein. Such antibodies may be obtained according to conventional methods using the whole mutant protein or partial proteins including the altered site of the mutant protein as an antigen. Any type of antibody including monoclonal and polyclonal antibodies and fragments thereof (e.g., Fab, F(ab′)2, Fv, scfv), as well as naturally occurring and modified antibodies (chemically modified, conjugated and chimeric antibodies), may be used for the detection of the proteins in the present invention, so long as the antibody binds to the mutant protein to be detected.

Alternatively, since HP1-alpha was found to bind to the mutant-LAMR1, LAMR1-TP1, but not to the naturally occurring LAMR1, the mutation in the amino acid sequence of the proteins (mutant-LAMR1 and HP1-alpha) can be determined by detecting the binding of HP1-alpha (or LAMR1) expressed in a ventricle of a subject to LAMR1 (or HP1-alpha). When binding of the HP1-alpha (or LAMR1) expressed in the subject to LAMR1 (or HP1-alpha) is detected, the amino acid sequence of the expressed HP1-alpha (or LAMR1) of the subject is determined to contain a mutation that may result in ARVD.

In the diagnosis methods of the present invention, the detection of a mutation in the amino acid sequence of LAMR1 or HP1-alpha in a subject can be carried out using, for example, tissues or cells of cardiac muscle, skeletal muscle and blood including leukocytes, cardiomiocytes and skeletal muscle cellsas the sample.

Any mammal may be diagnosed as the subject according to the present invention. Preferred mammalian subjects to be diagnosed by the present method include, human, mice, rats and dogs.

The above-described primers, probes and antibodies may be formulated as a diagnostic agent for ARVD. Such a diagnostic agent for ARVD comprises one or more of the above-described primers, probes and antibodies as the detection reagent. These detection reagents may be packaged in the form of a kit with other materials required for the diagnosis of ARVD (control reagent; means for detecting the primers, probes and/or antibodies; instructions; etc.). The detection reagents may be immobilized on a solid matrix (chip, etc.) as needed.

Animal Model

The present invention provides an animal model of ARVD. The present inventors found that mice with a mutation in Lamr1 gene inherit RVD. Thus, the present invention relates to a mutant Lamr1 gene-expressing animal that can be used as a model of ARVD.

Furthermore, the present invention revealed that injection of a mutant Lamr1 gene into a ventricle of a normal animal causes RVD in the animals. Thus, the present invention relates to a transgenic animal transduced with a polynucleotide expressing Lamr1 gene into a ventricle of the animal. Such transgenic animal can be used as an animal model of ARVD.

Lamr1-tp1 gene is a preferred example of the mutant Lamr1 gene expressed in the animal model or transgenic non-human animal of the present invention. However, the mutant Lamr1 gene is not restricted to Lamr1-tp1 gene. According to the present invention, HP1-alpha was shown to bind with LAMR1-TP1 but not with LAMRl. This binding of LAMR1-TP1 to HPl-alpha was demonstrated to influence transcriptional regulation of genes that are essential for the survival of cardiomyocytes, finally leading to lethal cell dysfunction. Thus, any gene may be used as the mutant Lamr1 gene expressed in the animal model or transgenic non-human animal of the present invention so long as it encodes a protein that binds to HP1-alpha and influences the transcriptional regulation of genes that are essential for the survival of cardiomyocytes. Such genes include, in addition to Lamr1-tp1, genes encoding LAMR1-TP1 wherein one or more amino acids are added, deleted, inserted and/or substituted, or genes highly homologous to the Lamr1-tp1 gene (e.g., an identity of 70%, 80%, 85%, 90%, 95%, or higher). The binding ability to HP1-alpha of a protein encoded by a mutant Lamr1 gene can be confirmed by contacting the protein with HP1-alpha and detecting the binding between the two. For example, a library that comprises genes serving as candidates of mutant Lamr1 gene of the present invention and displays the expression products of the genes on the surface can be used to detect a protein binding to HP1-alpha and obtain gene encoding the protein. Such libraries include phage libraries, and libraries comprising transformed cells or ribosomes expressing proteins on their surface. Methods for constructing these kinds of libraries are known in the art. For example, a library that comprises candidate mutant Lamr1 genes can be constructed according to conventional methods by amplifying the Lamr1-tp1 gene so that amplified products include random mutation(s) in their sequences, and the amplified products are incorporated into appropriate vectors for the library.

Both the animal models and the transgenic animals of the present invention include vertebrates other than human, and preferably are non-human mammals. Preferred examples of the animal models or transgenic animals of the present invention are rodents (e.g., mice and rats), and most preferably mice. However, in addition to mice and rats, transgenic animals of cats, cows, dogs, hamsters, goat, guinea pigs, pigs rabbits, sheep, and so on have been reported, and are included in the present animal models and the transgenic animals. Furthermore, since ARVD model animals are reported for dogs, cats and minks (Bright, J. M. and McEntee, M., J. Am. Vet. Med. Assoc 207: 64-66 (1995); Simpson, K. W. et al., J. Vet. Intern. Med. 8:306-309 (1994); Fox, P. R. et al., Circulation 102: 1863-1870 (2000); Ishikawa, S. et al., Arch. Pathol. Lab. Med. 101: 388-390 (1977)), transgenic animals of these species are particularly preferred

The animal models of the present invention may be transgenic animals or naturally occurring animals that express the mutant Lamrl gene. For example, the KK/Rvd mice accidentally discovered by the present inventors during the screening of anti-diabetic compounds using KK obese mice is included in the animal model of the present invention.

In addition, the animal models of the present invention include animals expressing artificially introduced mutant Lamr1 gene. The mutant Lamr1 gene may be inserted into an expression vector and directly injected into a ventricle of an animal. It is preferred to inject the expression vector into the right ventricle of an animal. Any method may be employed for the injection of the expression vector so long it allows the expression of the mutant Lamr1 gene. For example, the expression vector may be injected according to the method of Lin, H. et al. (Circulation 82: 2217-2221 (1990)).

Any expression vector may be successfully employed to produce the animal models of the present invention so long as the mutant Lamr1 gene is expressed in a ventricle, preferably the right ventricle, of an animal injected with the vector. In addition to the mutant Lamr1 gene, the expression vector comprises expression regulatory sequence(s) required for the expression of the gene and, if needed, marker gene(s) and such.

Furthermore, the animal models of the present invention include transgenic animals expressing the mutant Lamr1 gene. Both transgenic animals systemically expressing the mutant Lamr1 gene and those expressing the gene only in the heart or a ventricle (suitably in the right ventricle) are included in the animal models of the present invention. Limited expression of the mutant Lamr1 gene can be achieved by linking the gene downstream of a promoter that directs specific expression of the gene in the heart. Such promoters includes alpha-MHC promoter used in Example of the present specification. However, the present invention is not limited thereto, and it is obvious to those skilled in the art that any promoter of a heart (or ventricular)-specifically expressed gene may be used as the promoter to express the Lamrl gene in the animal models or transgenic animals of the present invention. In addition to the promoter, a recombinant gene construct to introduce the mutant Lamr1 gene into animals may include: other sequences required for the expression of the introduced gene, marker gene(s), and so on.

Methods to produce transgenic animals are well known in the art, and may be produce according to the literature (e.g., Proc. Natl. Acad. Sci. USA 77: 7380 (1980); Nature 385: 810 (1997); Nature 394: 369 (1998)). Specifically, a recombinant gene construct comprising the mutant Lamr1 gene is transduced into unfertilized eggs, fertilized eggs, sperms, embryonic cells including their primordial cells, cultured cells of embryonic cells (e.g., ES cells), etc., to develop the cells into individual animals. Animals wherein the transduced DNA is integrated into the somatic cells and germ cells are selected from the resulting animals. Any cell may be used to produce an animal model or transgenic animal of the present invention so long as it can be transformed with the mutant Lamr1 gene and can be developed as individual animals. The DNA encoding mutant Lamr1 gene can be transduced via the aggregation method, the calcium phosphate method, the DEAE-dextran method, electroporation, lipofection, microinjection, particle bombardment, etc. Alternatively, a transgenic animal can be prepared by fusing a cell transformed with the mutant Lamr1 gene with an embryonic cell.

More specifically, transgenic animals can be produced as follows. First, male pronuclei are transduced with recombinant gene construct comprising the mutant Lamr1 gene by microinjection. Generally, a higher copy number of integrated heterologous gene leads to increased gene expression. Thus, it is preferred to integrate multiple copies of the gene in tandem at the same site of the genome, when it is required to achieve a higher expression level. Then, eggs are fertilized using the pronuclei to obtain successfully transduced eggs. The fertilized eggs can be tested, for example, by PCR using specific primers or Southern blotting with specific probe to comprise the object gene in the correct orientation. The eggs are transplanted into the oviduct of a pseudopregnant foster mother, and born transgenic chimera animals are tested for the integration of the mutant Lamr1 gene. The transgenic chimera animals identified to comprise the mutant Lamr1 gene are mated with normal animas to obtain F1 animals. Among the F1 animals, animals containing the mutant Lamr1 gene in somatic cells (heterozygotes) are selected. Such selected F1 animals are crossed to obtain F2 homozygous animals.

The transgenic animal of the present invention may be of any generation of the transgenic animals described above, including the heterozygotes, so long as it expresses the mutant Lamr1 gene.

Cell Comprising Mutant Lamr1 Gene

According to an in vitro study, early death and mutation in the chromatin architecture were observed in cardiomyocytes expressing mutant Lamr1 gene. The early cell death and mutation in the chromatin architecture seem to be the cause of ARVD in animals expressing mutant Lamr1 gene. Therefore, cells introduced with the mutant Lamr1 gene find use in screening for compounds that serve as candidates of pharmaceuticals for treating or preventing ARVD.

Any cell may be used as the cells with a mutant Lamr1 gene of the present invention, so long as it expresses the gene and shows changes in the chromatin architecture or have shortened cell life. Any cell derived from animals or established cell lines may be transformed with the mutant Lamr1 gene. Preferred cells of the present invention include cardiomyocytes and cells derived from cardiomyocytes.

Any expression vector may be successfully employed to produce the cells of the present invention so long as the mutant Lamr1 gene is expressed in the cells. In addition to the mutant Lamr1 gene, the expression vector comprises expression regulatory sequences) required for the expression of the gene and, if needed, marker gene(s) and such. For example, the CA promoter may be used for directing the expression of the mutant Lamr1 gene and GFP may be used as the marker. Virus vectors, such as adenovirus vector, may be used as the expression vector of the present invention, but are not limited thereto. The vectors can be introduced into cells by general methods known to those skilled in the art, and as needed, the mutant Lamr1 gene may be introduced into the chromosome of the cell via homologous recombination.

Alternatively, the cells of the present invention may be derived from the above-described animal models or transgenic animals of the present invention. Methods known in the art can be used to establish cell lines from the animal models or transgenic animals of the present invention.

Screening Method

Compounds that alleviate the symptom in ARVD animal models are expected to function as pharmaceuticals to treat or prevent ARVD. Thus, the present invention provides a method for screening a compound using the ARVD animal model of the present invention.

Specifically, a compound for treating or preventing ARVD can be screened by: (1) administering a test compound to an ARVD animal model, (2) detecting symptoms of RVD in the animal; and selecting the compound that was detected to alleviate or suppress a symptom of RVD.

Any of the above-described animal models and transgenic animals of the present invention may be used as the ARVD animal model of the present screening method. The ARVD animal model may be at any disease stage of ARVD.

More specifically, if the ARVD animal model used in the screening method of the present invention is at a very primary stage without any notable symptom, the occurrence of ARVD may be detected in the animal. Compounds that suppress the occurrence of ARVD in such animals are expected to serve as prophylactic pharmaceuticals of ARVD. According to the invention, degenerative process of ARVD in mice with Lamrl-tpl gene started in the 6th week. Thus, the occurrence of ARVD may be confirmed, for example, by detecting fibrosis, calcification, degradation of cells and/or macrophage infiltration in the right ventricle of the ARVD animal model after breeding the animal model for more than 6 weeks. Furthermore, mice with Lamr1-tp1 gene were revealed to show prolonged QRS duration by electrocardiography. Therefore, electrocardiography may be taken for the ARVD animal model to detect the occurrence of ARVD.

Alternatively, if the ARVD animal model used in the screening method of the present invention is at a more progressed stage, compounds that alleviate or suppress a symptom of RVD may be searched. Such compounds may not serve as prophylactic pharmaceuticals of ARVD but are expected to find use in treating ARVD at more progressed disease stages.

According to the method, the test compound may be administered to the animal model parenterally or orally. Parenteral administration of the test compound include application to the skin, and injection and infusion into the blood vessel, muscle or lesion, but are not limited to these examples. For example, when the test compound is a protein, it may be administered using a vector, such as virus vector, as a gene encoding the protein. To administer the test compounds to the animal model in the present screening, they may be solubilized in sterilized or unsterilized buffer, water, isotonic liquids (e.g., saline, glucose solution, etc.), and such as needed. Furthermore, a test compound may be administered once, several times, or continuously during the screening of the invention.

Another embodiment of the screening method of the present invention uses a cell expressing a mutant Lamr1 gene of the present invention. Early death and alteration in the chromatin architecture were observed in cardiomyocytes expressing the mutant Lamr1 gene, and the changes seemed to be the cause of ARVD in animals expressing the mutant Lamr1 gene. Therefore, compounds that suppress or impede such changes in cells expressing the mutant Lamr1 gene are expected to function as pharmaceuticals to treat or prevent ARVD.

Specifically, cell(s) expressing a mutant Lamr1 gene is cultured under the existence of a test compound. The test compound may be added into the culture media at once, separated in several times, or continuously. Alternatively, when the test compound is a protein or polypeptide that is encodable by a gene, then the gene encoding the test compound may be inserted into an expression vector and transformed into the cell for the screening.

Then, changes in the cell(s) are observed. The changes in the cell(s) may be detected by comparing the cell number or chromatin architecture of the cell(s) cultured under the existence of a test compound with that of cell(s) cultured without the test compound.

Before the detection of changes, the culture of the cell(s) is continued for a sufficient time. According to the Example, the structural change of chromatin was observed in cell 10 to 12 hr after transfection of the gene, and the decrease in cell number occurred 24 hr after the transfection. Thus, when detecting changes in the chromatin structure as an index of the present screening, it is preferred to continue the culture of cell(s) for at least 10 hr. In case of counting cell numbers as an index of changes in the gene, it is preferred to continue the culture of cell(s) for at least 24 hr. By counting the cell numbers, the test compounds can be estimated whether they have an ability to prolong the life of cells expressing the mutant Lamrl gene.

Moreover, test compounds that may serve as a pharmaceutical to treat or prevent ARVD can be screened using changes in gene expression of cell(s) expressing the mutant Lamr1 gene as an index. This method can be performed similarly to the right above-described method by culturing cell(s) expressing mutant Lamr1 gene under the existence of a test compound, and then detecting changes in gene expression in the cultured cells. Also similarly to the above-described method, changes may be detected by comparing the result with that of cell cultured without the test compound.

In this method, it is necessary to select an appropriate time point to see the direct influence of the mutant Lamr1 gene expressed in the cell. For example, as shown in the Example (see, under the item of “7. Changes of gene expression induced by LAMR1-TP1”), when CA promoter was used for the expression of the mutant Lamr1 gene, LAMR1-TP1 was expressed 10 hr after transfection of the gene. Thus, to examine changes due to the mutant-LAMR1, the detection should take place after the expression of the mutant Lamrl gene. The time point when the mutant-LAMR1 is expressed largely depends on the used expression regulatory sequence. However, one skilled in the art can readily determine the time point when the mutant-LAMR1 is expressed. For example, as described in the Example, detectable marker protein, such as GFP, may be expressed together with the LAMR1, and the expression of GFP may be detected as an index of LAMR1 expression.

Methods for detecting differentially expressed genes between cells are known in the art. For example, changes in gene expression in the cultured cells can be detected using arrays of polynucleotides. Any array can be used so long as it allows detection of some genes that are differentially expressed in a normal cell and a cell expressing the mutant Lamr1 gene. Commercially available arrays may also be used. Specifically, total mRNA is prepared from the cell(s) cultured with and without a test compound, labeled (e.g. fluorescent label), and contacted with an array, respectively. The fluorescent intensity on the arrays are measured and compared to detect differentially expressed genes.

Alternatively, differentially expressed genes between cells can be detected hybrid subtraction. According to the method, differentially expressed genes can be selectively concentrated and cloned. Specifically, total mRNA is prepared from the cell(s) cultured with or without a test compound to prepare a cDNA sample. Then the cDNA sample is contacted with total mRNA (or cDNA) prepared from the other cultured cell(s). For example, this step may be performed by binding the first cDNA sample to a carrier, and then contacting the other total mRNA (or cDNA) sample. mRNA or cDNA that did not bind to the cDNA in the first cDNA sample can be separated as differentially expressed genes. Furthermore, a method called representation difference analysis (RDA) with higher sensitivity and allows specific detection is known in the art and can be used in the present screening.

The genes that are determined to be differentially expressed among the cells may be further confirmed by quantitative PCR. Furthermore, the sequence of the genes may be determined to provide an insight to the pathological role of the mutant-LAMR1.

As another embodiment of the present invention, a method of screening for a compound using binding between mutant-LAMR1 and HPl-alpha as an index is provided. According to the present invention, LAMR1-TPl was revealed to have an increased affinity for HP1-alpha and the interaction of LAMR1-TP1 and HP1-alpha seemed to be the cause of early cell death of cardiomyocytes. Thus, compounds that inhibit binding of a mutant-LAMR1 and HP1-alpha are expected to function as pharmaceuticals for treating or preventing ARVD. Specifically, the mutant-LAMR1 is contacted with HP1-alpha in the presence of a test compound, and then the test compound that inhibits binding between the two molecules is selected.

Any HP1-alpha may be used for the present screening so long as it binds to mutant-LAMR1, and in addition to LAMR1-TP1, any mutant-LAMR1 that retains the binding ability to HP1-alpha may be used. Both of the proteins to be used for the screening may be a recombinant polypeptide, derived from the nature, or a partial peptide thereof; or they may be in the form of purified polypeptide, soluble protein, bound to a carrier, or protein fused with other polypeptides so long as they retain the binding ability to each other.

The present screening can be carried out both in vitro and in vivo.

According to an in vitro assay, mutant LAMR1 or HP1-alpha is immobilized on a carrier, and the other protein is added with a test compound to the carrier. Then, the mixture is incubated for a sufficient time to form a complex between the mutant LAMR1 and HP1-alpha, washed and the other protein bound on the carrier is detected and/or measured.

Any substances may be used as the carrier so long as it does not inhibit the binding of the mutant LAMR1 and HP1-alpha, and may be in the form of beads, plates, fibers, etc. made of polysaccharides, resins and such. For example, if magnetic beads are used as the carrier, they can be readily collected and isolated using magnet.

A protein can be bound to the carrier through conventional methods including chemical bonding and physical adsorption; binding with the use of antibodies specifically recognizing the protein or by means of avidin and biotin.

Binding of the mutant LAMR1 and HP1-alpha can be performed in a buffer. It is preferred to choose a buffer that does not inhibit binding between the two proteins.

The detection and/or measurement of the other protein bound on the carrier can be performed using a biosensor utilizing the surface plasmon resonance phenomenon (e.g., BIAcore (Amersham Biosciences)). The interaction between the two proteins, mutant LAMR1 and HP1-alpha, can be followed real-time as a surface plasmon resonance signal, with small amount of proteins without label.

Alternatively, the detection and/or measurement can be performed by labeling the other protein and detecting the label on the carrier. Labels such as enzymes, fluorescent substances, and radioisotopes may be used. The enzyme labels may be detected by adding a substance and measuring the changes of the substrate due to the activity of the enzyme label. On the other hand, fluorescent substance labels can be detected using a fluorophotometer, and radioisotope labels may be detected by liquid scintillation, using a Geiger counter, etc. Furthermore, the protein may be labeled with biotin to detect the forming of a complex (mutant LAMRl/HPl-alpha) via avidin. Moreover, GST may be added to the protein, and the complex formed between the mutant-LAMRl and HPl-alpha can be detected using gluthatione.

As an alternate method for detecting and/or measuring the other protein bound on the carrier, an antibody may be used. For example, antibodies specifically recognizing the other protein may be used to detect the existence of the protein on the carrier. Furthermore, the other protein may be linked with an immunologically detectable compound (epitope). An epitope can be introduced to a protein using a commercially available epitope-antibody system. For example, vectors that express fusion polypeptides with β-galactosidase, glutathione S-transferase, green fluorescence protein (GFP), maltose-binding protein, etc. via a multi-cloning site are known in the art. It is preferred to introduce a small epitope to minimize the influence of the compound on the binding of the proteins. Preferred examples include E-tag, HA, His-tag, HSV-tag, myc, FLAG, T7-tag, VSV-GP, etc. Antibodies binding to these epitopes are commercially available and can be used for detecting the other protein bound on the carrier.

When an antibody is used for the detection and/or measurement, it is labeled with any of the labels mentioned above and the labels are detected. Alternatively, an antibody may be detected using a secondary antibody with a label, or protein G or protein A column.

Alternatively, GST may be added to either the mutant-LAMR1 or HP1-alpha, and the complex formed between the mutant-LAMR1 and HP1-alpha can be collected using glutathione (GST pull down method).

Two-hybrid system is also known as a method to detect binding between proteins (Cell 68: 597 (1992); Trends Genet. 10: 286 (1994)). According to the system, SRF-binding region is fused to either of the proteins and GAL4-binding region is fused to the other protein. These fusion proteins are expressed in yeast cells under the existence of a test compound. Without any inhibition of the test compound, binding of the two fusion proteins activates a reporter gene (e.g., Ade2 gene, CAT gene, HIS3 gene, lac Z gene, luciferase gene, etc.; any gene expressing detectable products can be used), and as a result positive clones can be detected.

Test compounds that may be used in the present screening methods include single compounds, such as inorganic compounds, natural products, organic compounds, proteins and peptides; compounds of various libraries including gene libraries and chemical compound libraries; cell culture supernatants; cell extracts; fermented products; marine organism extracts; plant extract; etc.

The following examples are presented to illustrate the present invention and to assist one of ordinary skill in making and using the same. The examples are not intended in any way to otherwise limit the scope of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. Any patents, patent applications and publications sited herein are incorporated by reference.

### EXAMPLE

Methods

1. PWK Mouse Strain

The PWK strain belonging to Mus musculus musculus subspecies separated from Mus musculus domesticus some 1 million years ago, and is maintained as one of the wild-type-derived inbred strains.

2. Lamr1-tp1 Expressional Analysis

PCR assays were performed to confirm the presence of each identified mutation. Mismatch assays for the 287T to C and 291G to T mutants of the nucleic acid sequence of Lamr1 introduced changes at the penultimate 3′ position for the forward primer and 868C to T for the reverse primer, respectively

Following primers were used for the assay:

| | |
|---|---|
| Lamr1 forward primer: | TTGCCATCGAGAATCCTG, |
| Lamr1 reverse primer: | TGTGCACTCCAGTCTTCCG, |
| Lamr1-tpl forward primer: | TTGCCATAGAGAACCCTC, |
| and | |
| Lamr1-tpl reverse primer: | TGTGCACTCCAGTCTTCCA. |

Each PCR product was digested with NheI (specific for Lamr1-tp1 amplicon) to produce fragments of 334 bp and 279 bp; the Lamr1 amplicon was uncut.

3. Injection of Recombinant DNA In Vivo

Female C57BL/6 mice (8-weeks-old, 22-25 g) were anesthetized with a mixture of ketamine (100 mg/kg i.p.) and xylazine (5 mg/kg i.p.), incubated, and ventilated. Left lateral thoracotomy was performed to expose the beating heart, and 10 g of plasmid DNA in 100 μl of phosphate-buffered saline (PBS) containing 5% sucrose was injected into the right ventricular wall using 30-g needle. The animals were killed 3 weeks after injection, and histological staining was performed.

4. Transgenic Mice Models

Three kinds of targeting vectors were constructed under following promoters (Lamr1-tp1; KSCX and alpha-MHC; Lamr1: alpha-MHC). These targeting vectors were introduced into blastocyst (derived from CS7BL/6 Jcl mouse strain) by a standard pronuclear microinjection technique.

5. Preparation of Adenovirus

Replication-defective recombinant adenoviral vectors expressing Lamr1-tp1-ires-GFP and Lamr1-ires-GFP were prepared using adenovirus expression vector kit (Takara) following the manufacturing protocol. Specifically, Lamr1-tp1 and Lamr1 cDNA connected to ires-GFP sequence (Clontech) placed downstream of a CA promoter comprising a cytomegalovirus enhancer, a chicken β-actin promoter and rabbit β-globin polyA were respectively inserted into a cassette cosmid vector that contained an entire adenovirus type 5 genome except for the E1a, E1b, and E3 regions. A recombinant adenovirus was constructed by in vitro homologous recombination in HEK293 cells using this cosmid vector and the adenovirus DNA terminal-protein complex. The desired recombinant adenovirus was purified by ultracentrifugation through CsCl₂ gradient followed by extensive dialysis. The titer of the virus stock was assessed by plaque formation assay using the HEK293 cells. Cardiomyocytes were infected with the recombinant adenovirus vectors at a multiplicity of infection (MOI) of 5-100 plaque forming units per cell. The expressions of GFP and F-actin were assessed by Western blotting using 20 μg of myocardial protein lysate.

6. Primary Culture of Neonatal Rat Ventricular Myocytes and MTS Assay

Ventricular myocytes obtained from 1- or 2-day-old Wister rats were prepared and cultured overnight in Dulbecco's modified Eagle's medium (DMEM) containing 10% fetal bovine serum as described previously (Simpson, P. et al., Circ. Res. 51: 787-801 (1982)). Cytotoxicity was assessed using CellTiter 96 Aqueous One Solution Cell Proliferation Assay System (Promega, Tokyo, Japan). Rat cardiomyocytes were cultured in 96-well culture plates at a density of 3×10⁴ cells/cm². Forty-eight hours after the addition of adenovirus to the myocytes, MTS reagent (3-(4,5-dimethylthiazol-2-yl)-5-(3-carboxymethoxyphenyl)-2-(4-sulfophenyl)-2H-tetrazolium) was added to each well. After 1 hr incubation, optical absorbance at 490 nm was measured using a microplate reader. Cell viability was expressed as mean percentages for the absorbance at MOI5 with the standard deviations of absorbance.

7. Antibodies

Antibodies (anti-MCP-1 (Santa Cruz), anti-GFP, anti-myc-conjugate beads (Clontech), anti-V5 (Invitrogen), and anti-HP1-alpha (Upstate)) were used for each experiment. Polyclonal antibody FD4818 was derived from rabbits against amino acid sequence (RALNVLQMKEEDVFK) corresponding to amino acids 3 to 15 of LAMR1.

8. Identification of LAMR1-TP1 Binding Protein (RVAP27)

2.0×10⁵ of COS7 cells expressing either PCDNA3.1-myc-tagged-Lamr1 or PCDNA3.1-myc-tagged-Lamr1-tp1 (Invitrogen) were ³⁵S-metabolic labeled and lysed with 1 ml lysis buffer (20 mM Tris pH8.0, 5% acetonitrile, 5 mMEDTA, 1% NP40) and immunoprecipitated with anti-myc-antibody. Antibody-bound materials were separated by SDS-PAGE, and the radioactivity was detected by BAS imaging analyzer (Fuji). Fraction obtained from myc-antibody beads was injected onto a Phenyl-RPLC column (4.6×250 mm, Nakarai) equilibrated with 0.1% trifluoroacetic acid, 5% acetonitrile, and eluted with a linear gradient of 27 to 37% acetonitrile at a flow rate of 1 ml. The eluted fractions were lyophilized and separated by SDS-PAGE. Radioactivity was detected by BAS imaging system.

9. Large Scale Purification and Sequence Analysis of LAMR1-TP1 Binding Protein

1.0×10⁸ of COS7 cells expressing myc-tagged-Lamr1-1-tp1 were lysed with 200 ml lysis buffer and applied to 500 μl of anti-myc-antibody beads (Clontech). Antibody-bound materials were eluted with 0.1% trifluoroacetic acid and 5% acetonitrile. The eluted fraction was diluted 50 times with lysis buffer and was applied to Uno-Q anion exchange column (Bio-Rad). The column was equilibrated with 20M Tris and 5% acetonitrile at pH8.0, and bound materials were eluted with a linear gradient of NaCl (0-0.5M) at a flow rate of 1 ml/min. Five fractions around 0.3M NaCl elution were pooled and loaded on Phenyl-RPLC column (4.6×250 mm, Nakarai) equilibrated with 0.1% trifluoroacetic acid and 5% acetonitrile. A linear gradient of 27-37% acetonitrile was passed through the column at a flow rate of 1 ml to collect the eluted fractions. After separating the fractions by SDS-PAGE, RVAP27 was detected eluted into the same fraction as the radioactive LAMR1-TP1. 10 pmol purified RVAP was subjected to SDS-PAGE on 12% gel. After staining the gel with SyproRuby, 27 kDa band was cut and treated with trypsin. The tryptic digest was fractionated by nanoscale high performance liquid chromatography on a C18 column (0.1×50 mm). Two fractions were analyzed by direct N-terminal sequencing by Edman degradation using HP G1005 Protein Sequencing System. One fraction was analyzed on tandem mass spectrometer (Q-Tof2) equipped with nanoelectrospray ionization source. Positive ion tandem mass spectra were measured.

10. RNA Preparation and Hybridization to Oligonucleotide Arrays

Total RNA was isolated from viable mice or cultured neonatal cardiomyocyte derived from C57BL/6 mice. Affymetrix Gene Chip technology was performed as previously described (Lockhart, D. J. et al., Nat. Biotechnol. 14: 1675-1680 (1996)). Specifically, first, cDNA was synthesized from total RNA and annealed to T7-oligo-dT primer. Reverse transcription was performed with Superscript II reverse transcriptase. Double-stranded cDNA synthesis was performed using DNA polymerase I with appropriate reagents. Synthesis of biotin-labeled cRNA was performed by in vitro transcription using MEGAscript T7 IVT Kit (Ambion, Inc.). The cRNA was fragmented and hybridized to GeneChip Murine U74vA2 Array Set (Affymetrix) Hybridization, probe washing, staining and probe array scan were performed according to protocols provided by Affymetrix.

11. Real-Time PCR

Real-Time PCR was performed using TaqMan technology and ABI Prism 7700 Detection System (Applied Biosystems). Reactions (25 μl) were set up using the 2× Universal PCR Master Mix (Applied Biosystems), template cDNA, and adequate concentrations of primers and probes. Experiments were done in duplicate for all the samples. To standardize the quantity of 2 selected genes, glyceraldehyde-3-phosphate dehydrogenase (GAPDH) was used as the endogenous control reference. According to the microarray analysis performed by the present inventors, the expression level of GAPDH was stable and no significant difference was observed among all the 3 groups.

12. Data Analysis

GeneSpring 5.0 (Silicon Genetics) software was used for analyses. Global normalization was employed to all data of the 18 arrays with a combination of 3 steps: transforming negative values to 0.01, normalizing to 50th percentile per chip, and normalizing to median per gene. The data was filtered using a combination of signal confidence (‘present’ flag), fold change (1.5 to 2 times), minimum acceptable signal intensity (average difference >=50 in at least 1 of 3 groups), and statistical cut-off (P<0.05, student's t-test). Data are presented as mean or mean±SEM. One-way ANOVA with Tukey-Kramer exact probability test was used to test the differences among all the groups and the least-squares method was used for linear correlation between selected variables. P<0.05 was considered statistically significant.

Results

1. Mouse Model of ARVD

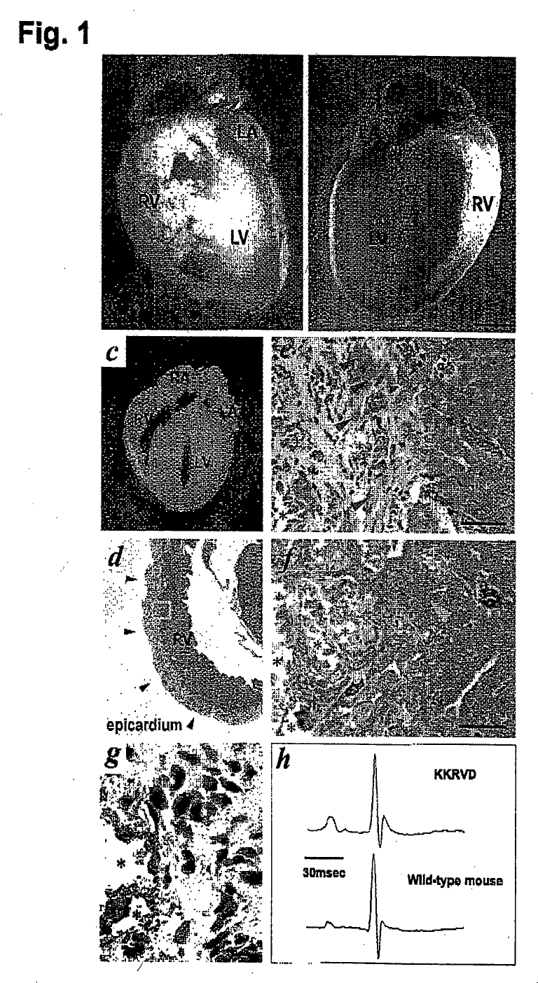

A mouse model of ARVD was accidentally discovered in the laboratory of the present inventors during the screening of anti-diabetic compounds using KK obese mice that were originally isolated on the basis of hyperglycemia (Kondo, K. et al., Bulletin of the Experimental animals 6: 107-112 (1967); Nakamura, M. and Yamada, K., Diabetologia 3: 212-221 (1967)). The strain of mice named KK/Rvd was found to develop severe RVD. In 8 week-old mice of this strain, massive fibrosis of the entire right ventricular (RV) wall that never extends to the left ventricle (LV) was revealed by macroscopic examination of the heart, which resembles the histopathology of human ARVD (FIGS. 1a and b). The outer one third of the RV wall of KK/Rvd mouse was replaced by fibrous tissue involving calcification (FIGS. 1c and d). This degenerative process starts in the 6th week and completes in 10 weeks. Some variations are observed in the distribution of affected cardiomyocytes. However, the penetrance of the phenotype was almost 100%. Histologically, degradation of cardiomyocytes and macrophage infiltration were observed at the border between the fibrosis tissue and the viable myocardial tissue (FIGS. 1e and g). This pathological finding suggests the progress of cardiomyocyte degeneration from outside to inside of RVD, and this is the most important pathological feature of ARVD in human (Burke, A. P. et al., circulation 97: 1571-1580 (1998)). Thin fibrous tissue was observed to surround the degraded cardiomyocytes at the edge of the myocardial degeneration (FIG. 1f), indicating progressive replacement by fibrous tissue. Lymphocyte infiltration was rarely observed even by immunohistochemical staining (data not shown). Autoimmune and infectious mechanisms were unlikely to be involved in the degeneration due to the lack of lymphocyte infiltration.

Detailed microscopic examination showed that the LV myocardium was completely intact and none of the changes detected in the RV myocardium could be observed. It is still unknown why RV is more susceptible than LV in these mice. Lamr1-tp1 injection into LV also induced cardiomyocyte degeneration and calcification. However, the changes were less severe (data not shown). In addition, transgenic mice expressing LAMR1-TP1 in both cardiac chambers showed predominant RV degeneration. The threshold of cardiomyocyte damage may be higher in LV compared with RV, and a higher level of Lamr1-tp1 gene expression may cause LV degeneration. In human ARVD, a part of LV is also often involved. Although both ARVD2 and Naxos disease are known to show RV-specific phenotype in human, responsible genes are equally expressed in both of the cardiac chambers (Tiso, N. et al., Hum. Mol. Genet. 10: 189-194 (2001); Protonotarios, N. et al., J. Am. Coll. Cardiol. 38: 1477-1484 (2001)). The mechanism leading to such biased RV susceptibility remains to be resolved. However, it may be explained by specific genes that determine the susceptibility to cell damage. LV cardiomyocytes are under high stress due to the high pressure in LV. Therefore, in the cardiomyocytes of LV, more cytoprotective genes can be supposed to be induced. In fact, microarray analysis comparing RV and LV has shown higher LV expression of genes categorized as genes related to cell and organism defense (Steenman, M. et al., Genomics 12: 97-112 (2003)). Dominant degeneration of the outer RV wall in ARVD also supports this concept. That is, the inner free wall is under more mechanical stress than the outer wall, and thus is expected to express more defensive genes, such as heat shock proteins. Thus, this mechanism might lead to LV protection in ARVD. Alternatively, RV susceptibility may be explained by the existence of RV-specific gene that is involved in the susceptibility of the right ventricle. For example, the lack of an RV-specific gene, actinin-associated LIM-domain protein, in mice is reported to cause ARVD-like features with somewhat different histological characteristics from human ARVD (Pashmforoush, M. et al., Nat. Med. 7: 591-597 (2001)).

Moreover, electrocardiography (ECG) revealed a prolonged QRS duration in KK/Rvd mice compared with wild-type mice (FIG. 1h). Electric conduction inhomogenity leading to QRS prolongation is also often seen in human ARVD patients (Peters, S. and Trummel, M., Ann. Noninvasive Electrocardiol. 8: 238˜245 (2003); Nasir, K. et al., Pacing Clin. Electrophysiol. 26: 1955-1960 (2003)). This result indicates an increase of susceptibility to arrhythmia caused by intraventricular conduction disturbance. However, tachyarrhythmia that is often seen in human ARVD could not be detected by the ECG monitoring. This may be due to the high beating rate and small size of mice heart, which is known to be difficult to generate lethal tachyarrhythmia (Cranefield, P., “The conduction of the Cardiac Impulse”, Futura Publishing Co., Inc., Mt Kisco, N.Y. (1975)).

Furthermore, other organs of these KK/Rvd animals showed no histological abnormalities. KK/Rvd mice matched three of the major clinical criteria for ARVD, i.e., 1) regional RV dysplasia, 2) inheritability, and 3) fibro-fatty replacement (McKenna, W. J. et al., Br. Heart J. 71: 215-218 (1994)). Thus, KK/Rvd mice were concluded to serve as a preferred model animal for human ARVD.

2. Identification of Right Ventricular Dysplasia Locus

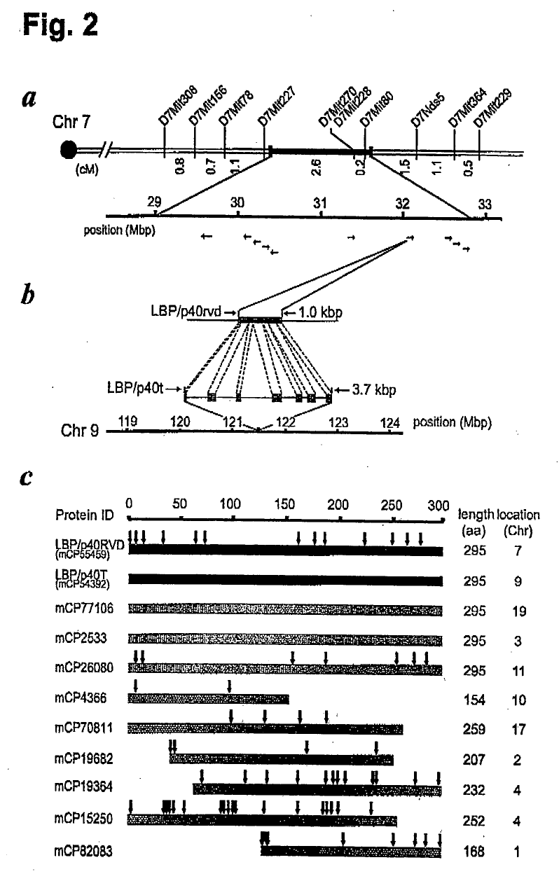

To investigate the mode of inheritance of RVD, cross test between wild-type PWK and KK/Rvd mouse strains was performed. F1 mice showed no RVD, while the segregation ratio of normal to RVD mice among the F2 and backcross progeny indicated that RVD was inherited as autosomal recessive trait. The locus responsible for RVD was dubbed “right ventricular dysplasia (rvd)”. Linkage analysis of these backcross mice (n=480) using 165 microsatellite markers revealed that the rvd locus was closely linked to D7Mit270 existing nearly on the middle of chromosome 7, with a maximum multipoint odds score of 4.67 (FIG. 2a). Using other markers that were deduced to flank with D7Mit270 from gene databases, these mice were further genotyped to localize the rvd locus to a region of approximately 3.0 cM.

Then the exons of this rvd locus were sequenced in the gene database to compare the sequences between KK/Rvd and PWK mice. Differences between the two strains newly discovered by this analysis were used as markers to narrow the candidate locus. Within the narrowed region (0.5 cM), a 1031 bp insertion absent in the PWK genome was found in the KK/Rvd genome (FIG. 2b). This insert was a 1031 bp retroposon that encoded mutant-Lamr1. The retroposon was dubbed “Lamr1-tp1 (laminin receptor 1, transposed paralog 1)”. There was neither an annotated area nor a dbEST matching area within about 1 Mb of this insertion, indicating that alteration of a nearby gene was not likely to be the cause of this phenotype and suggesting Lamr1-tp1 itself responsible for RVD. The original Lamr1 gene consists of 7 exons and 6 introns and is located on chromosome 9. Furthermore, it comprises 32 variants of retroposons that are probably derived from a retrovirus. The alignment of these paralog of Lamr1 is shown in FIG. 2c. Almost all of the retroposons have stop codons in the open reading frame and thus are unlikely to be translated. However, 4 Lamr1 retroposons, including Lamr1-tp1, have the stop codon in the same position as the Lamr1 cDNA. This suggests that these genes could be translated to produce proteins with various mutations. Among the 4 full-length retroposons, 2 genes have exactly the same sequence as Lamr1 and 2 genes encode mutant-Lamr1 (one is Lamr1-tp1 located on chromosome 7 in KK/Rvd mice and the other is located on chromosome 11). Lamr1-tp1 protein (LAMR1-TP1) shares 96% sequence identity with Lamr1 protein (LAMR1), and includes 13 amino acid mutations.

In humans, a highly conserved mutant-Lamrl has been isolated from a fetal brain cDNA library (Richardson, M. P. et al., Gene 206: 145-150 (1998)), suggesting that mutant-LAMR1 is also transcribed in human. Several reported human ARVD loci are located close to the retroposons of Lamr1 or histone-modulating protein genes (Table 1).

TABLE 1
Comparison of human LAMR1 and histone
related genes loci with ARVD loci

| ARVD candidate locus | | LAMR1 related gene | | Histone related gene | |
|---|---|---|---|---|---|
| ARVD 4 | 2q32.1- | XM_013127* | 2g31 | HAT 1 | 2q31.2- |
| | 32.3 | | | | 33.1 |
| ARVD 5 | 3p23 | LAMR1 | 3p21 | | |
| ARVD 6 | 10p12-14 | XM_053952* | 10p14 | | |

HAT 1: histone acetyl transferase 1,
*LAMR1 retroposon

3. Tissue Expression of Mutant-Lamr1 (Lamr1-tp1)

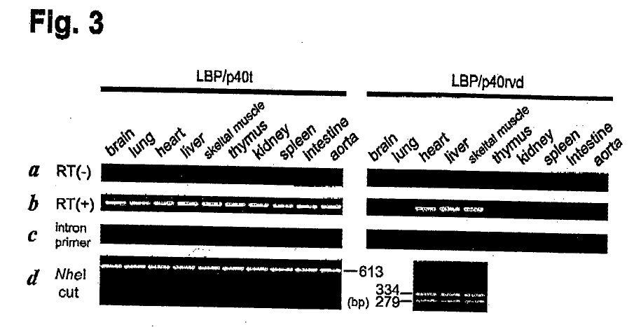

To be the cause of ARVD, this retroposon, Lamr1-tp1, must be transcribed in the hearts of KK/Rvd mice. Thus, Lamr1-tp1 and Lamr1 transcripts in the tissue of Lamr1-tp1 or Lamr1 transfected mice were detected by amplifying the transcripts via specific RT-PCR. Specifically, after isolation of total RNA, the samples were treated with DNase to eliminate contamination by genomic DNA before RT-PCR. Elimination of such contamination was confirmed through several PCR reactions using different pairs of intron primers. As a result, Lamrl-tpl mRNA was found to be only transcribed in the heart, liver, and skeletal muscle of KK/Rvd mice, unlike the ubiquitous expression of Lamrl mRNA (FIG. 3). Moreover, Lamr1-tp1 was not transcribed in any of the tissues of PWK mice or other wild-type mice (C57BL/6). RV and LV showed no difference in Lamr1-tp1 expression, suggesting the need of an additional factor to cause the specific pathological changes of ARVD. Despite the high expression of Lamr1-tp1 transcripts, no pathological changes were observed in the liver and skeletal muscle of KK/Rvd mice.

4. In Vivo Role of Mutant-LAMR1 (LAMR1-TP1)

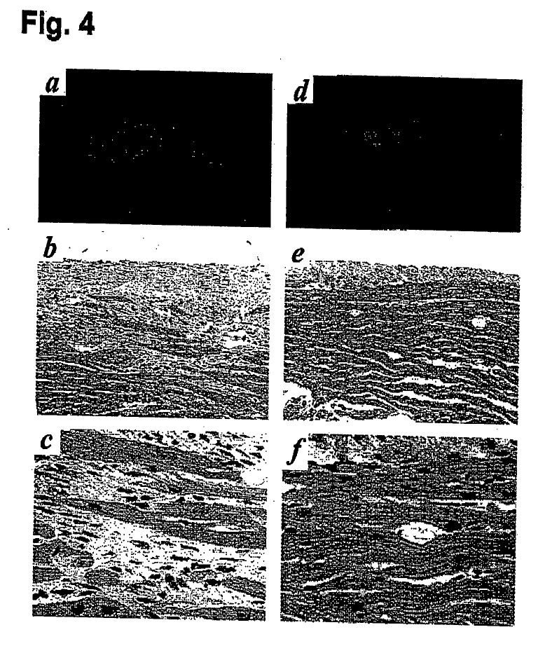

To confirm the responsibility of Lamr1-tp1 transcripts for the ARVD phenotype, functional studies of LAMR1-TP1 were performed, A GFP co-expression plasmid (pIRES2EGFP-Lamr1-tp1 or pIRES2EGFP-Lamr1) was transfected into the hearts of C57BL/6 mice by direct injection of DNA into the RV according to previously reported method (Lin, H. et al., Circulation 82: 2217-2221 (1990)). Three weeks later, LAMR1-TP1 expression was detected along with massive RV wall damage at the injected area. In mice injected with pIRES2EGFP-Lamrl-tp1, GFP-positive cardiomyocytes were observed in the zone of degeneration accompanied by fibrosis (FIG. 4a to c). The degeneration of transfected cardiomyocyte started 2 weeks after injection of the plasmid pIRES2EGFP-Lamr1-tp1. Lymphocyte infiltration was rarely seen in the injected area and similar changes were also observed in immunosuppressed SCID mice (data not shown). Therefore, this tissue damage was unlikely to involve autoimmunity. On the other hand, the hearts injected with plasmid pIRES2EGFP-Lamr1 only showed slight damage at the injection site and GPF-positive cells therein were healthy without degradation (FIG. 4d to f). Fibrous tissue was rarely seen in the Lamr1-transfected hearts.

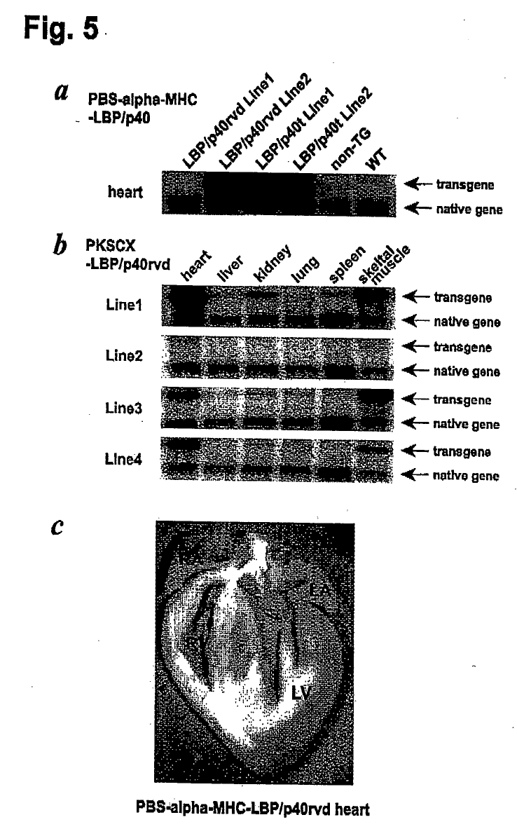

Then, the role of LAMR1-TP1 was further analyzed using transgenic mice. Two strains of transgenic mice were created. One systemically expressing Lamr1 (regulated by KSCX promoter) or expressing the gene only in the heart (alpha-MHC promoter). Furthermore, six substrains of Lamr1-tp1 transgenic mice (4 strains that express the gene under the KSCX promoter and 2 under the alpha-MHC promoter) and 4 substrains of Lamr1 transgenic mice were established (FIGS. 5a and b). Among the S strains of Lamrl-tpl transgenic mice, 4 strains showed cardiac expression of LAMRl-TPl.

All four strains expressed LAMRl-TP1 in the heart and showed extreme susceptibility to RV dysplasia (FIG. 5c). In some of the strains, tissue damage extended to the LV, however, RV involvement was always predominant in the tested strains. No phenotypic changes of other organs were detected in Lamr1-tp1 transgenic mice so far. In contrast, none of the strains expressing LAMR1 showed significant changes in any of their organs, including the heart. These data indicated that LAMR1-TP1 was responsible for RV dysplasia in KK/Rvd mice.

5. In Vitro Role of Mutant-LAMR1 (LAMR1-TP1)

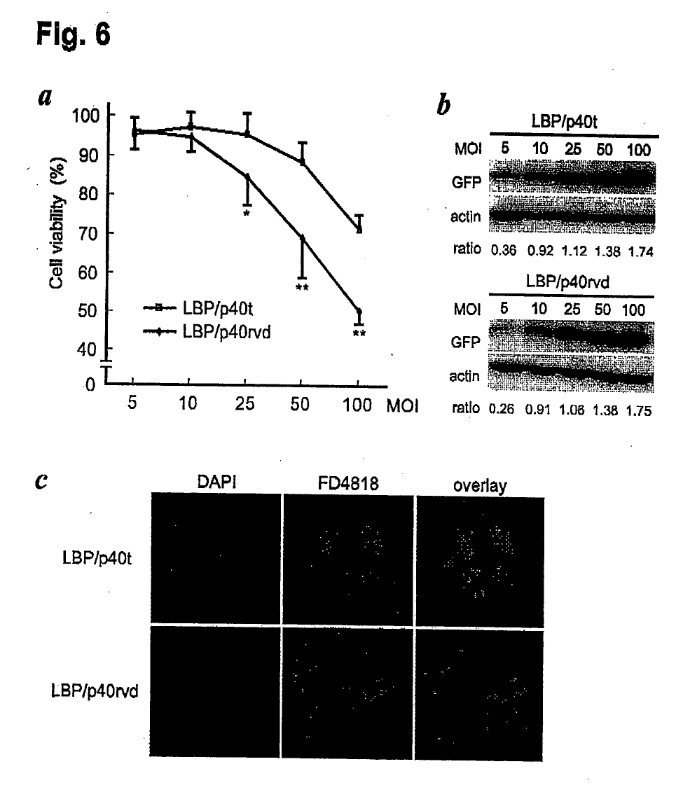

The in vitro expression of LAMR1-TP1 also caused impairment of cardiomyocytes function. Cultured rat cardiomyocytes were transfected with an adenovirus vector containing Lamr1-tp1-ires-GFP or Lamr1-ires-GFP under the control of a CA promoter. MTS assay was performed to clarify the effect of these constructs. Based on the expression level of GFP, with Lamrl-tp1-ires-GFP transfected cells and Lamr1-ires-GFP transfected cells showed similar transfection efficiency (FIG. 6b). However, only the expression of Lamr1-tp1 in cardiomyocytes led to a decrease in cell numbers at 48 hr after transfection (FIG. 6a). As a result of histochemical staining, the most prominent change in these cells was the alteration of the chromatin architecture. The staining of heterochromatin by DAPI showed a mosaic pattern in Lamr1-transfected cardiomyocytes. On the other hand, cardiomyocytes transfected with Lamr1-tp1 showed a speckled pattern of DAPI staining. Furthermore, the localization of LAMR1 and LAMR1-TP1 was analyzed by confocal microscopy. Specifically, rat cardiomyocytes transfected with adenovirus vector were stained using anti-LAMR1 antibody (FD4818). This antibody binds to both LAMR1 from LAMR1-TPl. However, endogenous LAMR1 was not detected due to its relatively low expression level. Interestingly, LAMR1 was identified in the DAPI negative euchromatin area of the nucleus, showing a mirror image to the pattern of DAPI staining (FIG. 6c, upper panel). On the other hand, transfection with Lamr1-tp1 altered the overall pattern of chromatin as described above and LAMR1-TP1 partially colocalized into the DAPI-positive heterochromatic loci (FIG. 6c, lower panel). These structural changes of chromatin were observed 10 to 12 hr after transfection and preceded the decrease in cell number that occurred 24 hr after transfection. The data suggested lethal effect of these chromatin changes on cardiomyocytes, although not fully excluding the possibility that these changes occurred secondary to lethal cell damage itself.

6. Binding of Heterochromatin Protein 1 to Mutant-LAMR1

To clarify the cellular mechanism of LAMR1-TP1 to cause conformational changes of heterochromatin, a protein that specifically interacts with LAMR1-TP1 was purified and cloned. The myc-tagged LAMR1-TP1 fusion protein expressed in ³⁵S-labeled COS7 cells showed the same migration pattern as myc-tagged LAMR1 (FIG. 7a). A 27 kDa protein (dubbed “RVAP27”) co-immunoprecipitated with LAMR1-TP1, but not with LAMR1. Using either a mouse cell line (3T3) or rat cardiomyocytes, RVAP27 was also shown to co-immunoprecipitate with transfected LAMR1-TP1 Large-scale purification of RVAP27 was performed by sequential column chromatography (FIG. 7b) to obtain about 10 pmol of RVAP27 from the lysate of 1.0×10⁸ COS7 cells (FIG. 7c). RVAP27 was digested, and obtained fragments were analyzed by Edman degradation N-terminal sequencing or nanoelectrospray ionization tandem mass spectrometry. As a result, RVAP27 was shown to include the amino acid sequences “SNFSNSADDIK”, “WKDTDEADLVLA”, and “CPQIVIAFYEER” that all matched with that of human HP1-alpha (FIG. 7d). Anti-myc antibody co-precipitated myc-tagged HP1-alpha with V5-tagged LAMRl-TPl, but not with V5-tagged LAMR1. This result verifies a specific interaction between HP1-alpha and LAMRl-TPl (FIG. 7e). HP1 is an important heterochromatin protein that regulates gene silencing through the interaction with methylated histones (Nakayama, J. et al., Science 292: 110-113 (2001)). HP1-alpha is also known to co-localize with DAPI-positive heterochromatin (Festenstein, R. et al., Science 299: 719-721 (2003)). HP1-alpha was confirmed to show the same staining pattern with DAPI dense region in cardiomyocytes expressing HP1-alpha (FIG. 7f). According to the immunohistochemical data (FIG. 6c), LAMR1 was located in the euchromatin area (DAPI negative) and LAMR1-TP1 was partially translocated to the heterochromatin area (DAPI positive). These findings imply that the mutant-LAMR1 had an increased affinity for HP1-alpha and thus was translocated to the heterochromatin. Such translocation might influence transcriptional regulation and interfere with the expression of genes essential for the survival of the cardiomyocytes, thus leading to lethal cell dysfunction due to LAMR1-TP1.

HP1-alpha is a key component of condensed DNA, and is involved in gene silencing by the interaction with methylated histone H3. Recently, mobility of HP1-alpha has been reported in various cells (Festenstein, R. et al., Science 299: 719-721 (2003); Cheutin, T. et al., Science 299: 721-725 (2003)). The stochastic competition of factors like LAMR1-TP1 with HP1-alpha may determine the fate of heterochromatin plasticity that is involved in regulating the fate of cells. Class II histone deacetylase (HDAC) has been reported to act as a signal-responsive suppressor of transcriptional events that govern cardiac hypertrophy and heart failure (Zhang, C. L. et al., Cell 110: 479-188 (2002)). HP1-alpha is known to bind with class II HDAC (Zhang, C. L. et al., Mol. Cell. Biol. 22: 7302-7312 (2002)), and thus may modify cardiac cell metabolism. Therefore, LAMR1-TP1 translated from an active retroposon in ARVD mice of the present invention seems to interact with HP1-alpha to lead the cardiomyocytes to early death.

Several reported human ARVD loci are located close to the retroposons of Lamrl or histone-modulating protein genes (Table 1), suggesting that either LAMR1 or HP1 may cause hereditary RVD in humans. Thus, analysis of LAMR1 and HP1 mutations at these loci in ARVD patients may potentially help to develop therapeutic tools for this lethal disease.

7. Changes of Gene Expression Induced by LAMR1-TP1

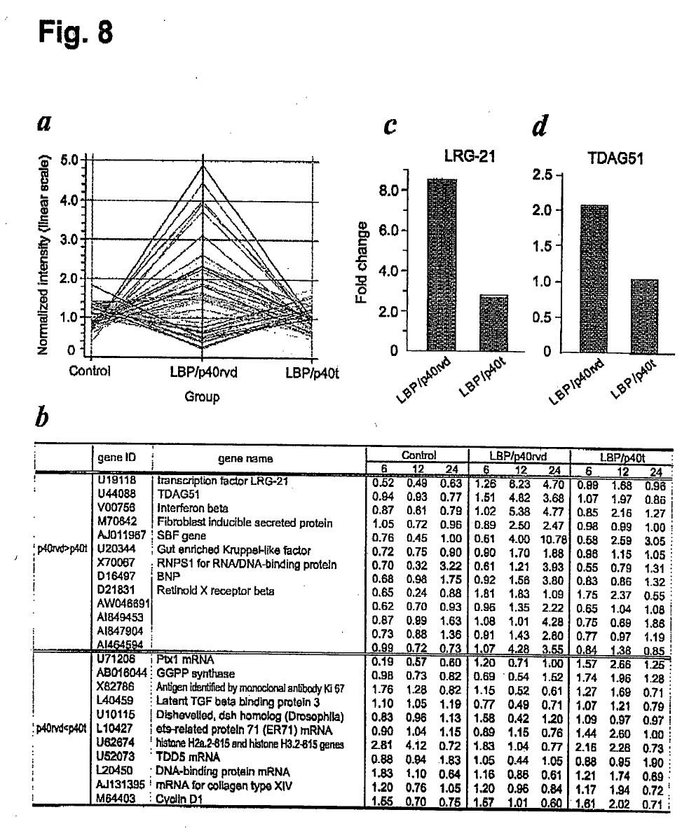

To investigate transcriptional regulation by LAMR1-TP1, changes in gene expression of cultured cardiomyocytes were analyzed after transfection of Lamr1-tp1 or Lamr1. Cells expressing Lamr1-tp1 are dying 24 hr after transfection. Therefore, gene expression at 6, 12, and 24 hr (each in duplicate) were analyzed to exclude the secondary effects of lethal cell damage. As a result, GFP expression indicated that the transfected protein was expressed 10 hr after transfection, indicating that the expression of genes at 6 hr was largely induced by viral infection itself. Interestingly, wild-type LAMR1 had minimal effect on the expression profile compared with that of non-transfected cardiomyocytes even though the adenovirus vector itself should cause some cell damage (FIG. 8a). On the other hand, LAMR1-TP1 caused substantial changes of gene expression at 12 and 24 hr, but not at 6 hr (FIG. 8b). These genes showed similar changes in expression in the duplicate analyses. The expression of these genes was further confirmed by quantitative PCR. (FIGS. 8c and d). The results suggested that LAMR1-TP1, but not LAMR1, could alter the expression of some specific genes. To understand the pathological role of LAMR1-TP1, the involvement of these genes in determining the fate of cardiomyocytes remains to be investigated.

While the invention has been described in detail and with reference to specific embodiments thereof, it will be apparent to one skilled in the art that various changes and modifications can be made therein without departing from the spirit and scope of the invention.

1. A method for diagnosing arrhythmogenic right ventricular dysplasia (ARVD), which comprises the steps of:
(1) detecting mutation in the amino acid sequence of a protein encoded by laminin receptor 1 (Lamr1) gene or retroposon thereof in a subject; and
(2) when a mutation is found in step (1), determining the subject to be susceptible to ARVD.

2. The method of claim 1, wherein the mutation in the amino acid sequence of the protein encoded by Lamr1 gene is determined based on the nucleotide sequence of the genomic Lamr1 gene of the subject.

3. The method of claim 1, wherein the mutation in the amino acid sequence of the protein encoded by Lamr1 gene or retroposon thereof is determined based on the nucleotide sequence of the expressed mRNA of the subject.

4. The method of claim 1, wherein the mutation in the amino acid sequence of the protein encoded by Lamr1 gene or retroposon thereof is determined based on the amino acid sequence of the expressed Lamr1 protein (LAMR1) of the subject.

5. The method of claim 1, wherein the mutation in the amino acid sequence of the protein encoded by Lamr1 gene or retroposon thereof is determined by detecting the binding of the expressed LAMR1 of the subject to heterochromatin protein 1 alpha (HP1-alpha), and when binding of LAMR1 to HP1-alpha is detected, the amino acid sequence of protein encoded by the Lamr1 gene is determined to contain mutation.

6. A method for diagnosing ARVD, which comprises the steps of:
(1) detecting a mutation in the amino acid sequence of protein encoded by Hpl-alpha gene of a subject; and
(2) when a mutation is found in step (1), determining the subject to be susceptible to ARVD.

7. The method of claim 6, wherein the mutation in the amino acid sequence of the protein encoded by Hp1-alpha gene is determined based on the nucleotide sequence of the genomic Hp1-alpha gene of the subject.

8. The method of claim 6, wherein the mutation in the amino acid sequence of the protein encoded by Hp1-alpha gene is determined based on the nucleotide sequence of the expressed mRNA of the Hp1-alpha gene of the subject.

9. The method of claim 6, wherein the mutation in the amino acid sequence of the protein encoded by Hp1-alpha gene is determined based on the amino acid sequence of the expressed HP1-alpha of the subject.

10. The method of claim 6, wherein the mutation in the amino acid sequence of the protein encoded by Hp1-alpha gene is determined by detecting the binding of the expressed HP1-alpha of the subject to LAMR1, and when binding of the Hp1 protein to LAMR1 is detected, the amino acid sequence of the protein encoded by Hp1-alpha gene is determined to contain mutation.

11. A diagnostic agent for ARVD, which comprises at least a substance that enables the detection of a mutation in the amino acid sequence of the protein encoded by Lamr1 gene of a subject compared to a naturally occurring sequence.

12. The diagnostic agent of claim 11, which comprises an antibody against a mutant LAMR1 as the substance.

13. The diagnostic agent of claim 11, which comprises HP1-alpha as the substance.

14. The diagnostic agent of claim 11, which comprises a probe against Lamr1 gene as the substance.

15. The diagnostic agent of claim 11, which comprises primers that can be used for specifically amplifying Lamr1 gene as the substance.

16. A diagnostic agent for ARVD, which comprises at least a substance that enables the detection of a mutation in the amino acid sequence of the protein encoded by Hp1-alpha gene of a subject compared to a naturally occurring sequence.

17. The diagnostic agent of claim 16, which comprises an antibody against a mutant HP1-alpha as the substance.

18. The diagnostic agent of claim 16, which comprises LAMR1 as the substance.

19. The diagnostic agent of claim 16, which comprises a probe against Hp1-alpha gene as the substance.

20. The diagnostic agent of claim 16, which comprises primers that can be used for specifically amplifying Hp1-alpha gene as the substance.

21. An animal model of ARVD, expressing a mutant Lamr1 gene.

22. The animal model of claim 21, wherein ARVD is caused by the injection of a mutant Lamrl gene into a ventricle.

23. The animal model of claim 22, wherein the gene is injected into the right ventricle.

24. The animal model of claim 21, wherein the animal is a transgenic animal expressing a mutant Lamr1 gene in a ventricle.

25. The animal model of claim 24, wherein the mutant Lamr1 gene is expressed in the right ventricle.

26. The animal model of claim 21, wherein the animal is a mouse.

27. A transgenic non-human animal transduced with a polynucleotide that expresses a mutant Lamr1 gene.

28. The transgenic non-human animal of claim 27, wherein the animal is a rodent.

29. The transgenic non-human animal of claim 28, wherein the animal is a mouse.

30. The transgenic non-human animal of claim 27, wherein the animal shows symptoms of ARVD.

31. The transgenic non-human animal of claim 27, wherein the mutant Lamr1 gene is Lamr1-tp1.

32. The transgenic non-human animal of claim 27, wherein the animal expresses the Lamr1 gene in a ventricle.

33. The transgenic non-human animal of claim 27, wherein the animal expresses the Lamrl gene in the right ventricle.

34. A cell expressing a mutant Lamr1 gene.

35. The cell of claim 34, wherein the cell is a cardiomyocyte.

36. A method of screening for a compound using the animal of claim 21 or 27, which comprises the steps of:
(1) administering a test compound to the animal of claim 21 or 27;
(2) detecting symptoms of right ventricular dysplasia (RVD) in the animal;
(3) selecting the compound detected as alleviating or suppressing a symptom of RVD.

37. A method of screening for a compound using the cell of claim 34, which comprises the steps of:
(1) culturing the cell of claim 34 in the existence of a test compound; and
(2) selecting the compound that prolongs the life of the cell or impedes changes in the chromatin architecture compared to a cell cultured in the absence the test compound.

38. A method of screening for a compound using the cell of claim 34, which comprises the steps of:
(1) culturing the cell of claim 34 in the existence of a test compound; and
(2) selecting the compound that suppresses changes in gene expression due to the introduction of Lamr1-tp1.

39. A method of screening for a compound using mutant-LAMR1 that binds to HP1-alpha and HP1-alpha, which comprises the steps of:
(1) contacting the mutant-LAMR1 and HP1-alpha in the presence of a test compound; and
(2) selecting the compound that inhibits binding between the mutant-LAMR1 and HP1-alpha.


SEQUENCE LISTING (9 sequences)

SEQ ID NO: 1 - DNA, 18 bp, Artificial
  Lamr1 forward primer
ttgccatcgagaatcctg

SEQ ID NO: 2 - DNA, 19 bp, Artificial
  Lamr1 reverse primer
tgtgcactccagtcttccg

SEQ ID NO: 3 - DNA, 18 bp, Artificial
  Lamr1-tp1 forward primer
ttgccatagagaaccctc

SEQ ID NO: 4 - DNA, 19 bp, Artificial
  Lamr1-tp1 reverse primer
tgtgcactccagtcttcca

SEQ ID NO: 5 - protein, 191 aa, Artificial
  RVAP27
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 6 - protein, 12 aa, Artificial
  RVAP27
WKDTDEADLVLA

SEQ ID NO: 7 - protein, 12 aa, Artificial
  RVAP27
CPQIVIAFYEER

SEQ ID NO: 8 - protein, 11 aa, Artificial
  RVAP27
SNFSNSADDIK

SEQ ID NO: 9 - protein, 15 aa, Artificial
  LAMR1 Amino Acids 3-15
RALNVLQMKEEDVFK